(19) United States
(12) Patent Application Publication
(10) Pub. No.: US 2019/0054194 A1
(43) Pub. Date: Feb. 21, 2019

(54) System and Method For Evaluation of Subjects Using Magnetic Resonance Imaging and Oxygen-17

(71) Applicant: Case Western Reserve University, Cleveland, OH (US)

(72) Inventors: Xin Yu, Pepper Pike, OH (US); Ciro Ramos-Estebanez, Cleveland, OH (US); Yuchi Liu, Cleveland, OH (US); Yuning Gu, Cleveland, OH (US); Yifan Zhang, Cleveland, OH (US); Chris Flask, Cleveland, OH (US)

(21) Appl. No.: 16/103,316

(22) Filed: Aug. 14, 2018

Related U.S. Application Data
(60) Provisional application No. 62/546,995, filed on Aug. 17, 2017.

(51) Int. Cl. A61K 49/06; A61B 5/055; G06T 7/11; G06T 7/168; A61B 5/00; G01R 33/56; G01R 33/48

(52) U.S. Cl. CPC A61K 49/06; A61B 5/055; G06T 7/11; G06T 7/168; A61B 5/7257; A61B 5/0042; A61B 2576/026; G01R 33/4824; G06T 2207/10096; G06T 2207/20056; G06T 2207/30016; G06T 2207/30104; G01R 33/5601

(57) ABSTRACT

A system and method for evaluating subjects using MRI and a contrast agent that overcomes the low sensitivity nature of previous detection methods is provided by using a 3D golden-angle-based radial sampling approach. In one configuration, direct detection of metabolic H₂¹⁷O generated from mitochondrial respiration may be imaged. Radial encoding allows for the use of ultra-short echo-time to compensate for signal loss due to the short T₂ relaxation time of ¹⁷O and other contrast agents. In addition, the golden-ratio-based sampling scheme has the flexibility of enabling various undersampling schemes and retrospective selection of temporal resolution for dynamic imaging. A 3D radial sampling scheme may also give rise to additional SNR gain by further shortening the echo-time.

### CROSS-REFERENCE TO RELATED APPLICATIONS

This application claims the benefit of U.S. Provisional Patent Application Ser. No. 62/546,995 filed Aug. 17, 2017 and entitled “SYSTEM AND METHOD FOR EVALUATION OF SUBJECTS USING MAGNETIC RESONANCE IMAGING AND OXYGEN-17.”

### STATEMENT REGARDING FEDERALLY SPONSORED RESEARCH

N/A

### BACKGROUND

MRI with arterial spin labeling uses magnetically labeled endogenous water as a tracer to measure parameters such as cerebral blood flow (CBF). Other imaging methods have used isotope-labeled exogenous water tracers to quantify both CBF and water transport across the Blood-brain-barrier (BBB). Using water labeled with oxygen isotopes, oxygen-15 (¹⁵O) positron emission tomography (PET) and oxygen-17 (¹⁷O) MRI, are the only available methods capable of tracing water movement in vivo. However, ¹⁵O-PET requires an on-site cyclotron to generate the short-lived ¹⁵O isotope (approximately a 2 minute half-life), limiting its clinical use. Alternatively, ¹⁷O is a stable MR-detectable isotope with a natural abundance of 0.037% only. ¹⁷O-labeled water (H₂¹⁷O) can thus serve as a tracer for in vivo evaluation of water movement by MRI. However, use of ¹⁷O-labeled water in vivo is challenging due to the limited signal-to-noise ratio (SNR) caused by the low natural abundance and MR sensitivity of ¹⁷O. Even on high-field scanners, images are typically acquired with a large voxel size to achieve adequate SNR. In addition, using a short echo time to minimize signal loss in ¹⁷O-MRI is critical due to the short transverse relaxation time of ¹⁷O.

Regulation of cerebral fluids plays a vital role in brain function. Investigation of cerebral fluid dynamics, such as blood flow, diffusion, and water movement across the blood-brain barrier (BBB), is of great value in assessing brain physiology and function under normal and diseased conditions. ¹⁷O-MRI has been used to evaluate cerebral metabolic activity in rats, healthy cats, and mice. Quantification of the cerebral metabolic rate of oxygen consumption (CMRO₂) was accomplished by calculating the rate of H₂¹⁷O generation from inhaled ¹⁷O-labeled oxygen gas (¹⁷O₂). Water dynamics and CBF can also be assessed with ¹⁷O-MRI by observing the kinetics of H₂¹⁷O signal either from a bolus injection of ¹⁷O-labeled water or from metabolically produced H₂¹⁷O from inhaled ¹⁷O₂. Most of these studies used spectroscopic imaging methods with Cartesian encoding, but alternative ¹⁷O-MRI methods have used ultrashort echo time sequences with non-Cartesian encoding, including the density-adapted three-dimensional (3D) radial pulse sequence and the flexible twisted projection imaging acquisition. These methods have achieved a spatial resolution of 8 to 8.5 mm and a temporal resolution of 40 to 50 s in humans at high field. However, such spatial and temporal resolution is not sufficient for imaging small rodents and lacks the SNR required for a clinical deployment of ¹⁷O-MRI to evaluate cerebral metabolic activity.

Ischemic stroke remains the most common cause of disability worldwide. Reperfusion may salvage the tissue and lead to functional and neurological improvement. Thrombolytic therapy using recombinant tissue plasminogen activator (rtPA) may be beneficial when administered within 3 hours of the onset of stroke. Recent analyses of pooled data from six randomized trials also suggest that the beneficial effect of rtPA may extend beyond 3 hours, although the risk of intracranial hemorrhage also increases. Hence, imaging methods that can provide accurate assessment of the salvageable penumbra and the risk for hemorrhagic transformation are critically important to extend the therapeutic time window of rtPA for maximized benefit and minimized risk.

Magnetic resonance imaging (MRI) plays an important role in post-stroke management by providing quantitative and region-specific assessment of the brain. In particular, perfusion-weighted MRI (PWI) is effective in identifying regions with perfusion deficits, while diffusion-weighted MRI (DWI) is sensitive to cytotoxic edema. Lesions identified by DWI are often considered to represent the infarct core, and regions with a PWI deficit yet normal DWI are regarded as the potentially salvageable penumbra. Hence, areas with perfusion-diffusion mismatch have been considered representing salvageable tissue that require active treatment. Fundamentally, penumbra and ischemic core are defined by metabolic parameters. Although perfusion-diffusion mismatch aims to identify two different metabolic regions, it does not quantify metabolic activity directly because the critical parameter for brain metabolism is the cerebral oxygen consumption rate (CMRO2) rather than blood flow. However, direct quantification of CMRO2 remains largely elusive. Although positron emission tomography (PET) using short-lived oxygen-15 (15O) radioactive tracers allows the estimation of CMRO2, the requirement of an on-site cyclotron for radio-isotope generation has greatly limited its clinical applications.

Hemorrhagic transformation is a complication of rtPA treatment. Emerging evidence suggests that breakdown of the blood-brain barrier (BBB) is a critical component in the development of hemorrhagic transformation. Hence, determination of BBB integrity in the acute stage of stroke is highly beneficial to predicting the risk of thrombolytic treatment. However, current imaging methods that rely on the extravasation of relatively large molecular contrast agents as an indicator of BBB leakage are only sensitive to a severely damaged BBB. Sensitive detection of early-stage BBB disruption remains an unmet need.

Inspired by ¹⁵O-PET studies, oxygen-17 (¹⁷O) MRI methods have been developed to quantify cerebral metabolism in vivo. Following the inhalation of ¹⁷O-oxygen (¹⁷O₂), CMRO₂ can be calculated by tracking the generation of ¹⁷O-water (H₂¹⁷O) in mitochondria, the last step in oxidative metabolism. In addition to metabolic assessment, ¹⁷O-MRI can also be used to directly quantify water exchange across BBB by tracking the extravasation of H₂¹⁷O injected intravenously. These noninvasive approaches do not require the use of radioactive tracers or toxic contrast agents. Hence, they are readily applicable to clinical patients.

Due to the low sensitivity of ¹⁷O, current use of ¹⁷O-MRI has been limited to high-field scanners with low spatial resolution. Alternatively, H₂¹⁷O can also be detected indirectly by taking advantage of the ¹⁷O-induced shortening in T₂ or T_{1ρ} of water proton (¹H). A major advantage of the indirect method is the high spatial resolution offered by ¹H-MRI. However, current T₂ or T_{1ρ} weighted methods either do not allow absolute quantification of H₂¹⁷O concentration, or lack the temporal resolution for dynamic tracking of H₂¹⁷O generation.

### SUMMARY OF THE DISCLOSURE

The present disclosure addresses the aforementioned drawbacks by providing a system and method for evaluating subjects using MRI and ¹⁷O that overcomes the low sensitivity nature of ¹⁷O detection by using a 3D golden-angle-based radial sampling approach for direct detection of metabolic H₂¹⁷O generated from mitochondrial respiration. Radial encoding allows for the use of ultra-short echo-time to compensate for signal loss due to the short T2 relaxation time of ¹⁷O. In addition, the golden-ratio-based sampling scheme has the flexibility of enabling various undersampling schemes and retrospective selection of an image update rate (temporal resolution) for dynamic imaging. In some embodiments, the undersampling schemes may be deployed in all three spatial dimensions. In some embodiments, by eliminating the phase-encoding gradient, the use of a 3D radial sampling scheme may also give rise to additional SNR gain by further shortening the echo-time.

In one configuration, a method is provided for ¹⁷O imaging using a magnetic resonance imaging (MRI) system. The method includes administering ¹⁷O to a subject and acquiring MR data for a region of interest containing the ¹⁷O within the subject using the MRI system. A k-space filter is applied to the MR data to create filtered MR data by: i) segmenting k-space into at least one region; ii) determining a number of spokes for the k-space region based upon a radial sampling pattern; and iii) determining a spoke spacing for the number of spokes in the radial sampling pattern using a golden-ratio. An MR image of the ¹⁷O within the subject is reconstructed using the filtered MR data.

In some configurations, the method includes determining a number of spokes using a Fibonacci sequence to uniformly distribute the spokes in at least one k-space region. The number of spokes may also be determined for at least two k-space regions and the Fibonacci sequence may determine a different number of spokes for a second k-space region than the number of spokes in the first k-space region. The number of spokes in the k-space region may be selected to fulfill a Nyquist sampling criterion.

In some configurations, the k-space filter may be applied to a 2D region of k-space and k-space may be segmented into at least one region where the 2D region of k-space may take the form of a ring. The spoke spacing in this configuration may be determined by an azimuth angle spacing divided by the golden-ratio. Segmenting k-space may include segmenting k-space into 5 ring regions. In the configuration where 5 ring regions are established, the number of spokes for the 5 ring regions may include 21, 34, 55, 89, or 144 spokes.

In some configurations, the k-space filter may be applied to a 3D region of k-space and k-space may be segmented into at least one region where the 3D region of k-space may take the form of a sphere. The spoke spacing may be determined by using both an azimuth angle spacing divided by the golden-ratio and a polar angle spacing divided by the golden-ratio.

In some configurations, reconstructing an MR image may include regridding and Fourier transforming the filtered MR data, and/or may include reconstructing a dynamic MR image series. Acquiring the MR data may include sampling k-space using a radially sampling trajectory. In some configurations, a temporal resolution achieved by performing the method may be within a temporal response window of ¹⁷O. The ¹⁷O may be administered to the subject via enriched water or oxygen gas.

In one configuration, a method is provided for contrast agent imaging using a magnetic resonance imaging (MRI) system. The method includes administering a contrast agent to a subject and acquiring MR data for a region of interest containing the contrast agent within the subject using the MRI system. A k-space filter is applied to the MR data to create filtered MR data by: i) segmenting k-space into at least one region; ii) determining a number of spokes for the k-space region based upon a radial sampling pattern; and iii) determining a spoke spacing for the number of spokes in the radial sampling pattern using a golden-ratio. An MR image of the contrast agent within the subject is reconstructed using the filtered MR data.

In one configuration, a system for contrast agent imaging is provided that includes a contrast agent administered to a subject. The system also includes a magnetic resonance imaging (MRI) system configured to acquire MR data for a region of interest containing the contrast agent within the subject. A computer system is also included and configured to apply a k-space filter to the MR data to create filtered MR data by: i) segmenting k-space into at least one region; ii) determining a number of spokes for the k-space region based upon a radial sampling pattern; and iii) determining a spoke spacing for the number of spokes in the radial sampling pattern using a golden-ratio. An MR image of the contrast agent within the subject is reconstructed using the computer system and the filtered MR data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a block diagram of an example magnetic resonance imaging (“MRI”) system that can implement the methods described in the present disclosure.

FIG. 2 is a depiction of data selection scheme for a time frame.

FIG. 3 is a depiction of k-space segmented into a set of rings.

FIG. 4 is a depiction of k-space for a 3D implementation of the present disclosure.

FIG. 5 is a flowchart showing one configuration for creating a k-space filter according to the present disclosure.

FIG. 6 is a flowchart showing one configuration for an application of the present disclosure to contrast imaging.

### DETAILED DESCRIPTION

In one configuration, the present disclosure provides a system and method for evaluating subjects using MRI and a contrast agent that overcomes the low sensitivity nature of previous detection methods by using a 3D golden-angle-based radial sampling approach. In one configuration, direct detection of metabolic H₂¹⁷O generated from mitochondrial respiration may be imaged. Radial encoding allows for the use of ultra-short echo-time to compensate for signal loss due to the short T₂ relaxation time of ¹⁷O and other contrast agents. In addition, the golden-ratio-based sampling scheme has the flexibility of enabling various undersampling schemes and retrospective selection of an image update rate (temporal resolution) for dynamic imaging. In some embodiments, the undersampling schemes may be deployed in 2D or in all three spatial dimensions. In some embodiments, by eliminating the phase-encoding gradient, the use of a 3D radial sampling scheme may also give rise to additional SNR gain by further shortening the echo-time.

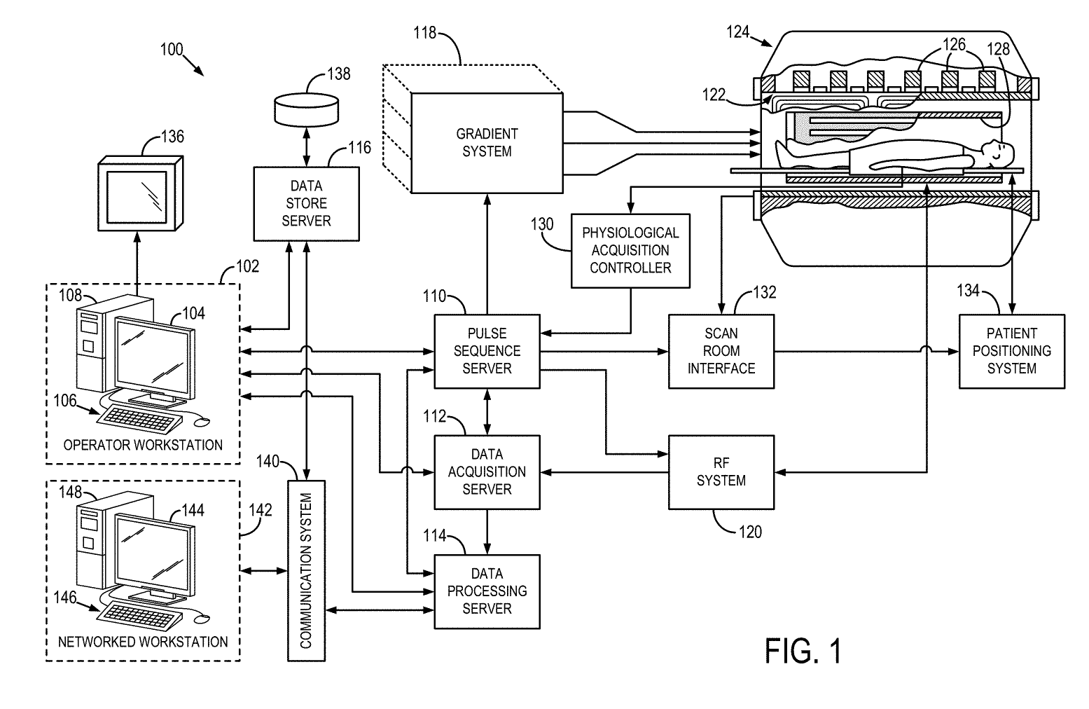

Referring particularly now to FIG. 1, an example of an MRI system 100 that can implement the methods described here is illustrated. The MRI system 100 includes an operator workstation 102 that may include a display 104, one or more input devices 106 (e.g., a keyboard, a mouse), and a processor 108. The processor 108 may include a commercially available programmable machine running a commercially available operating system. The operator workstation 102 provides an operator interface that facilitates entering scan parameters into the MRI system 100. The operator workstation 102 may be coupled to different servers, including, for example, a pulse sequence server 110, a data acquisition server 112, a data processing server 114, and a data store server 116. The operator workstation 102 and the servers 110, 112, 114, and 116 may be connected via a communication system 140, which may include wired or wireless network connections.

The pulse sequence server 110 functions in response to instructions provided by the operator workstation 102 to operate a gradient system 118 and a radiofrequency (“RF”) system 120. Gradient waveforms for performing a prescribed scan are produced and applied to the gradient system 118, which then excites gradient coils in an assembly 122 to produce the magnetic field gradients Gₓ, G_{y}, and G_{z} that are used for spatially encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil to perform the prescribed magnetic resonance pulse sequence. Responsive magnetic resonance signals detected by the RF coil 128, or a separate local coil, are received by the RF system 120. The responsive magnetic resonance signals may be amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the prescribed scan and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. An RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 110 may receive patient data from a physiological acquisition controller 130. By way of example, the physiological acquisition controller 130 may receive signals from a number of different sensors connected to the patient, including electrocardiograph (“ECG”) signals from electrodes, or respiratory signals from a respiratory bellows or other respiratory monitoring devices. These signals may be used by the pulse sequence server 110 to synchronize, or “gate,” the performance of the scan with the subject's heart beat or respiration.

The pulse sequence server 110 may also connect to a scan room interface circuit 132 that receives signals from various sensors associated with the condition of the patient and the magnet system. Through the scan room interface circuit 132, a patient positioning system 134 can receive commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, so that data is not lost by data overrun. In some scans, the data acquisition server 112 passes the acquired magnetic resonance data to the data processor server 114. In scans that require information derived from acquired magnetic resonance data to control the further performance of the scan, the data acquisition server 112 may be programmed to produce such information and convey it to the pulse sequence server 110. For example, during pre-scans, magnetic resonance data may be acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also process magnetic resonance signals used to detect the arrival of a contrast agent in a magnetic resonance angiography (“MRA”) scan. For example, the data acquisition server 112 may acquire magnetic resonance data and processes it in real-time to produce information that is used to control the scan.

The data processing server 114 receives magnetic resonance data from the data acquisition server 112 and processes the magnetic resonance data in accordance with instructions provided by the operator workstation 102. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 114 are conveyed back to the operator workstation 102 for storage. Real-time images may be stored in a data base memory cache, from which they may be output to operator display 102 or a display 136. Batch mode images or selected real time images may be stored in a host database on disc storage 138. When such images have been reconstructed and transferred to storage, the data processing server 114 may notify the data store server 116 on the operator workstation 102. The operator workstation 102 may be used by an operator to archive the images, produce films, or send the images via a network to other facilities.

The MRI system 100 may also include one or more networked workstations 142. For example, a networked workstation 142 may include a display 144, one or more input devices 146 (e.g., a keyboard, a mouse), and a processor 148. The networked workstation 142 may be located within the same facility as the operator workstation 102, or in a different facility, such as a different healthcare institution or clinic.

The networked workstation 142 may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be remotely processed by a networked workstation 142.

In one configuration, a 2D stack-of-stars radial sampling method may be implemented based on a golden-angle acquisition scheme. In some configurations, a k-space-weighted image contrast (KWIC) reconstruction may be applied to the acquired data to improve the temporal rate with preserved spatial resolution. Because changes in image intensity are determined primarily by the low-frequency data in dynamic imaging, undersampled radial images still retain abundant dynamic information due to the intrinsic oversampling of the center k-space by the radial trajectory. The golden-ratio-based radial profile order can meet the demands for high temporal and spatial resolutions. Instead of sampling the k-space with evenly spaced radial lines in the order of increasing azimuthal angles from 0 to pi (or 2pi), a golden-ratio-based profile uses an azimuthal angle spacing of pi (or 2pi) divided by the golden ratio. This sampling scheme achieves a nearly uniform coverage of k-space with an arbitrary number of spokes that may be symmetrically arranged in k-space. In one configuration, k-space coverage becomes most uniform when the number of spokes reaches a Fibonacci number. The golden-ratio-based profile order is extremely flexible, enabling various undersampling schemes and retrospective selection of an image update rate/temporal resolution for dynamic imaging.

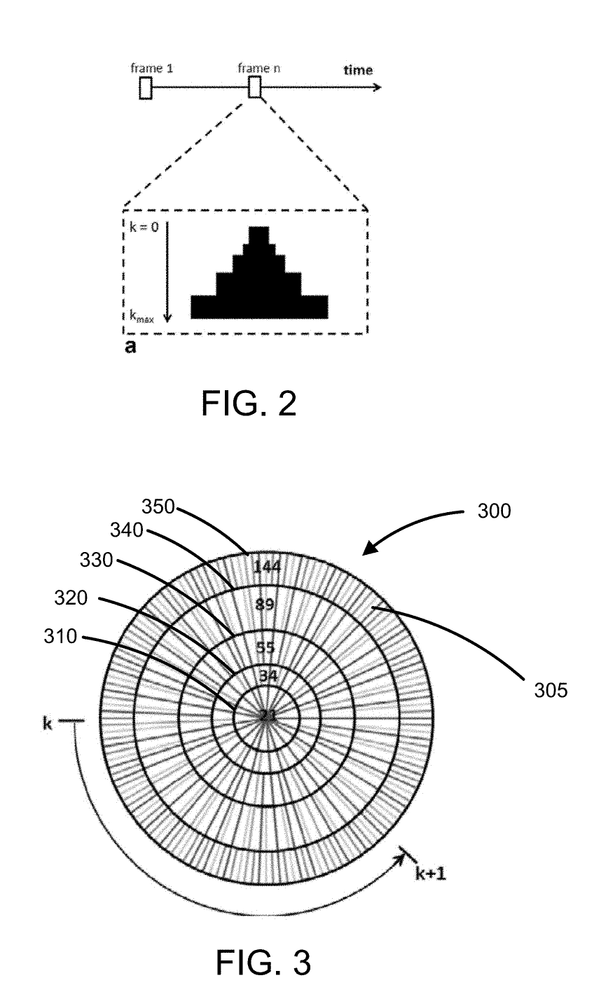

Referring to FIG. 2, data selection for a specific time frame for one configuration is shown. Half of the k-space from the center of k-space to kₘₐₓ is shown. The k-space filter may be shifted by a selected number of spokes from one time frame to the next. In one configuration, a dynamic golden-angle acquisition was combined with KWIC reconstruction to achieve high temporal resolution with preserved spatial resolution.

Referring to FIG. 3, a 2D data sampling method is depicted where, to reconstruct a specific time frame, k-space may be segmented into a set of rings. The rings, including the central ring 310, may consist of radial spokes 305 acquired at the time frame of interest. In one configuration, the number of spokes 305 in each ring may be a Fibonacci number and moving outwards from the central ring 310 the number of spokes 305 in each ring may be the next Fibonacci number in a sequence. In one configuration, image reconstruction may use a k-space filter 300 consisting of five rings with 21 spokes 305 for central ring 310, 34 spokes 305 for second ring 320, 55 spokes 305 for third ring 330, 89 spokes 305 for fourth ring 340, and 144 spokes 305 in outer ring 350. In this five ring example, the filtered data is of a total of 1228 data points in k-space. The radius of each ring may be chosen such that the number of spokes 305 in each ring fulfills a Nyquist sampling criterion. The filtered data may be regridded and Fourier transformed into images with a matrix size of 32×32, or other appropriate size, such as 64×64. Prior to regridding, the density compensation function may be calculated and applied to the data. For the example where the central ring 310 contains 21 spokes 305, this data sampling and reconstruction approach allows for an effective temporal resolution of 7.56 s (equivalent to the acquisition time of the central 21 spokes 305) with a voxel size of 11.25 μL (5.625 μL nominal), surpassing the spatial and temporal resolution achieved at ultrahigh fields. In one configuration after the k-space filtering, all images may be reconstructed using the nonuniform fast Fourier transform (NUFFT) toolbox.

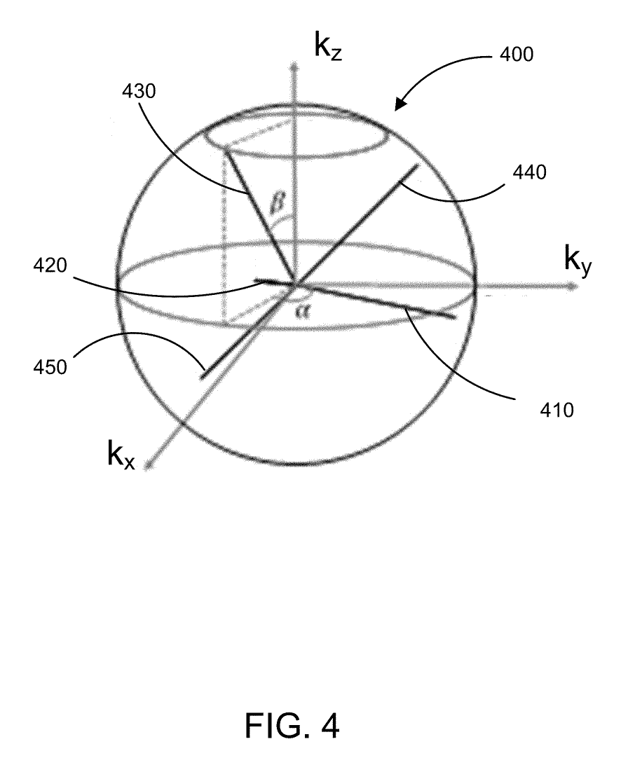

Referring to FIG. 4, a 3D data sampling method is depicted that allows for the flexibility of choosing a temporal window like the 2D golden-angle-based method. In this 3D radial sampling scheme, the polar and azimuth angles are determined by the multidimensional golden angle methods. In an example configuration depicted in FIG. 4, first spoke 410, second spoke 420, third spoke 430, fourth spoke 440, and fifth spoke 450 are shown in FIG. 4. It will be appreciated that any number of spokes may be used. In one configuration, the multidimensional golden angle may be derived from modified Fibonacci sequences. In one configuration, the golden-angle-based 3D radial sampling scheme uses an azimuth (β) and polar (α) angles for each spoke 410, 420, 430, 440, and 450, which are determined from 2D golden angles (φ₁ and φ₂). In one configuration, these parameters are represented by: k_{z}=2*{mφ₁}−1; β=cos⁻¹(k₂); α=2π*{mφ₂}

Relatively uniform distribution of the spokes on a unit sphere 400 can be achieved. “Relatively uniform” means the deviation in the distance between the spokes is minimized. This 3D radial sampling approach allows 3D radial spokes to distribute uniformly or relatively uniformly on a sphere, which may be a symmetric arrangement of spokes, and thus preserves the adaptive nature of image reconstruction to specific temporal windows. It allows 3D dynamic images to be reconstructed at any spatial or temporal resolution from the same dataset, and without the need for a priori knowledge of the exact balance between spatial and temporal resolution.

In some configurations of the 3D sampling method, every spoke samples the center of k-space, which will have additional SNR benefit over the stack-of-stars sampling scheme. In this configuration, when the k-space filter segments k-space into a different numbers of 3D shells in a similar manner to the 2D rings depicted for k-space in FIG. 3, the central shell will contain the greatest number of spokes since each spoke in the outer shells will sample the center of k-space.

In some configurations, the 3D sampling method uses the KWIC reconstruction method described above with the 2D sampling method. The KWIC reconstruction method in dynamic imaging is facilitated by combining it with a golden-ratio-based 3D radial sampling profile. An advantage of combining the golden-angle sampling and KWIC reconstruction is the flexibility to retrospectively choose the temporal rate for the reconstructed images. Golden-ratio-based 3D radial sampling enables quick updates of the center k-space so that no a priori knowledge of the expected kinetics is needed at the stage of data acquisition. A specific k-space filter can be chosen in postprocessing for optimal data reconstruction. In principle, any k-space filter that satisfies the Nyquist criterion can be used. With a golden-ratio-based sampling profile, both SNR and k-space coverage may be optimized when the number of spokes is a Fibonacci number. In one configuration, a k-space filter designed using Fibonacci numbers allows for satisfaction of the Nyquist criterion with a minimal number of spokes and may be optimized for dynamic studies that require high temporal resolution. When using a Fibonacci method, there may be flexibility in choosing the number of spokes for a specific temporal resolution, since the difference between two adjacent Fibonacci numbers increases gradually.

In one configuration of the 3D data sampling method, the center of the k-space filter uses 60 spokes. This configuration is able to achieve an isotropic spatial resolution of 1.5×1.5×1.5 mm³ (FOV, 24×24×24 mm³; matrix size, 16×16×16). With 20 signal averages for each spoke and a repetition time (TR) of 10 ms, a temporal resolution of 12 s can be achieved. Optimal k-space filter may be determined from retrospective reconstruction using point-spread-function as the criteria. The results may be compared to data acquired using the stack-of-stars sampling method with the same field of view and spatial resolution. SNR efficiency (SNR/\sqrt{Acquisition Time}) may also be used for comparison.

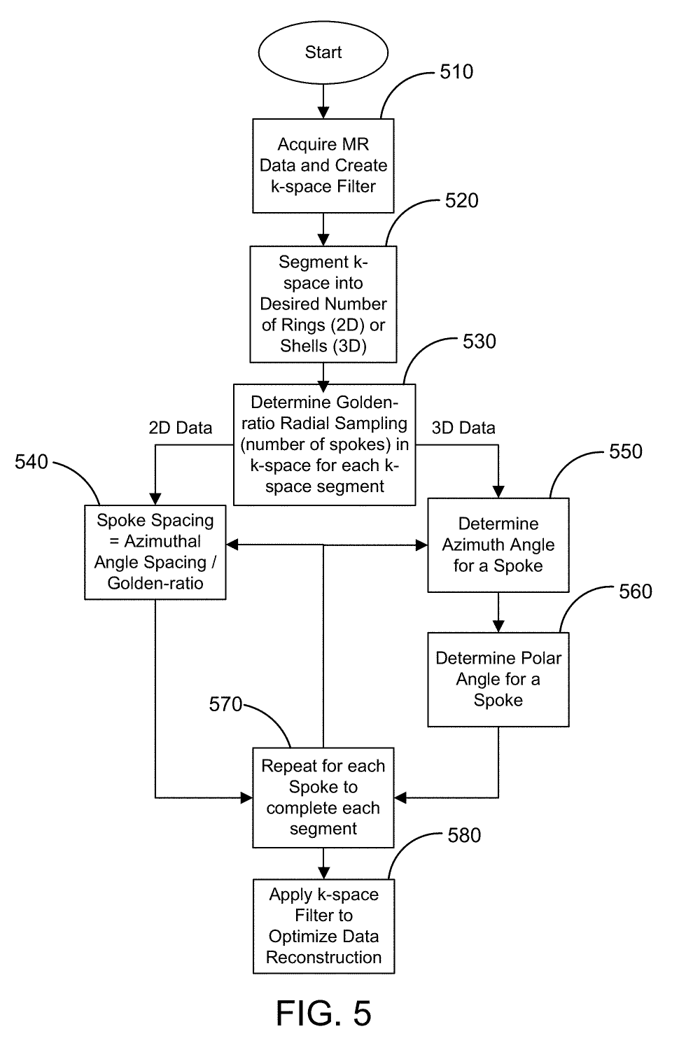

Referring to FIG. 5, a flowchart that depicts one configuration for creating a k-space filter is shown, where the process starts at step 510 with acquiring MR data and proceeds with creating a k-space filter thereafter. The k-space filter is created by segmenting k-space into a desired number of rings for 2D data processing, or a desired number of shells for 3D data processing at step 520. At step 530, the number of spokes for the filter is determined by using a golden-ratio based radial profile, where the number of spokes is determined for each of the k-space segments determined in step 520. For 2D data sampling, spoke spacing may be determined as the azimuthal angle spacing divided by a golden-ratio at step 540. For 3D data sampling, each spoke may have its azimuth angle and polar angle determined in a similar golden-ratio method at steps 550 and 560 respectively. The process of assigning spoke location or spacing is repeated to determine the location of each spoke in a k-space segment and is repeated for each k-space segment at step 570. The filter is completed by completing all of the spokes for all of the desired k-space segments and the completed filter is applied to optimize MR data reconstruction at step 580. A reconstructed image may be displayed to a user after the process is complete.

Clinical applications of the disclosed systems and methods include delineating altered water movement and oxygen metabolism in the brain and other organs under normal and diseased conditions, such as for detection of glioblastomas, brain tumors, dynamic hetero-nuclei imaging, cerebral oxygen consumption rate (CMRO₂) mapping, cerebral blood flow (CBF) mapping, oxygen extraction fraction (OEF) mapping, blood brain barrier (BBB) permeability assessment and structure evaluation, metabolic assessment in acute stroke, and the like, and may facilitate assessment of the risk and benefit of thrombolytic therapy for a subject. In each case, ¹⁷O may be administered intravenously, or by inhalation, or a combination of both either prior or during an imaging scan, or a combination of both.

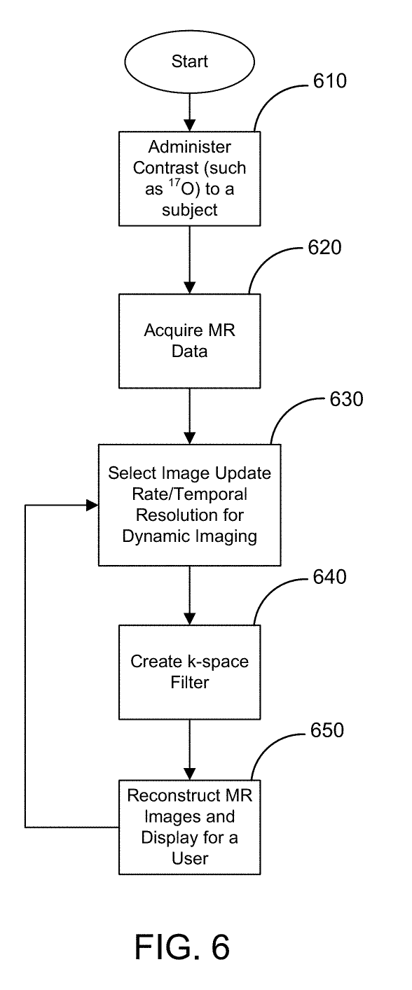

Referring to FIG. 6, a flowchart for one configuration of applying the above described k-space filters for ¹⁷O imaging is shown. ¹⁷O is administered to a subject at step 610. Administration of ¹⁷O can be through any appropriate methods, such as intravenous injection, inhalation, and the like. MR data is acquired at step 620 by using an MR imaging system. A user may choose to select or specify an image update rate or temporal resolution for a dynamic imaging application at step 630. A k-space filter is then created as described above and in FIG. 5 at step 640. Images are reconstructed and displayed for a user at step 650. The results of the reconstructed image may also be used to guide the creation of the k-space filter, such as to meet a need for a certain temporal resolution, in this way step 630 and the rest of the process may be repeated after step 650 until the desired result is achieved.

In one configuration, the dynamic ¹⁷O-MRI data may be used for the estimation of cerebral metabolic rate of oxygen consumption (CMRO₂) and cerebral blood flow (CBF). The sparsity of the parameter space may also be utilized to further accelerate data acquisition. Model-based compressed sensing methods for fast proton T₁ and T₂ mapping of a myocardium may be used for accelerating data acquisition. These methods may be adapted and combined with the 3D radial ¹⁷O-MRI method for fast quantification of CMRO₂ and CBF in an organ (such as the brain) with high spatial resolution. These combined efforts may allow for an isotropic voxel size of 3.375 μL (nominal) with a temporal resolution of 12 s.

In one configuration, tumor and brain may be differentiated with the present methods, such as to assess the presence of a glioblastoma. The kinetics of H₂¹⁷O uptake and washout in a brain with a glioblastoma (GBM) may be delineated after an intravenous bolus injection of H₂¹⁷O. ¹⁷O-MRI data may be used to show that the golden-angled-based ¹⁷O-MRI method provides the necessary temporal resolution to capture the rapid changes in ¹⁷O signal during the injection and washout phases, which allowed the differentiation of signal kinetics not only between tumor and normal brain tissues, but also between subjects with different tumor volumes. For example, the method may allow for differentiation between subjects with a moderate tumor volume (<30%, GBM-M) and those with a large tumor volume (>30%, GBM-L). Once image reconstruction has been completed, ¹⁷O images may be coregistered with the T₂-weighted ¹H images. Tumor areas may show higher signal intensity in T₂-weighted ¹H images due to having a longer T₂ than normal brain tissue. Appropriate regions of interest (ROIs) that encompass the tumor and normal brain tissue may be drawn manually from T₂-weighted ¹H images, or automatic segmentation routines may be used to define the ROIs. Total tumor and brain volumes may be calculated by adding the tumor and brain areas in all of the image slices. The percentage of tumor volume may then be calculated as the tumor volume divided by total brain volume.

¹⁷O signal may be normalized by the corresponding natural abundance ¹⁷O signal acquired before H₂¹⁷O injection or administration. Peak H₂¹⁷O uptake may be identified as the maximal signal intensity following H₂¹⁷O administration. A normalized time course of the ¹⁷O signal from peak uptake to the end of the measurement may be fit to a monoexponential function in a pixel-wise fashion to generate the maps of peak and steady-state H₂¹⁷O uptake, as well as its washout rate. Mean values for each ROI may be calculated for each map. In tissue with no obvious washout phase or even continued accumulation of H₂¹⁷O after the injection, washout rate may be set to zero, while peak and steady-state H₂¹⁷O uptake may be calculated as the normalized ¹⁷O signals immediately after H₂¹⁷O injection and at the end of measurement, respectively.

In an example experiment, control mice showed rapid uptake and washout of H₂¹⁷O, and GBM-M mice also showed similar ¹⁷O signal kinetics in normal brain tissue. In tumors, however, GBM-M mice exhibited reduced H₂¹⁷O uptake, while GBM-L mice showed no obvious washout phase, or even continuous accumulation of H₂¹⁷O after injection, suggesting a disrupted microcirculation system in tumors. In addition, GBM-L mice also showed significantly reduced H₂¹⁷O uptake in normal brain tissue with prolonged washout, a consequence of compromised cerebral perfusion due to tissue compression.

These experimental results demonstrate the feasibility of golden-angle-based ¹⁷O-MRI in tracking rapid changes of ¹⁷O signal during an injection study with high spatial and temporal resolution. This method has also been applied to ¹⁷O₂-inhalation studies on mice subject to MCAO surgery. One advantage of the golden-angle-based data sampling approach is the flexibility, or adaptiveness, of retrospective image reconstruction at any temporal window.

In one configuration, ischemic stroke imaging may be performed with the disclosed methods where a ¹H-based, high-resolution ¹⁷O-MRI method provides for sensitive detection of alterations in cerebral metabolism and BBB function in post-stroke brain. Although the T₂-shortening effect of ¹⁷O on water proton has been recognized, there still lacks a fast T₂ mapping method that can track H₂¹⁷O kinetics in vivo. The dynamic T₂ mapping method in the present configuration may be based on a magnetic resonance fingerprinting (MRF) technique. MRF uses an unconventional acquisition framework to create a unique signal evolution pattern (fingerprint) that is associated with tissue properties such as relaxation times, perfusion, diffusion, etc. A dictionary that encompasses all possible fingerprints is generated based on MR physics. By using pattern matching algorithms to select the dictionary entry that best matches the acquired fingerprint, efficient and simultaneous quantification of multiple tissue properties has been demonstrated, with drastically reduced sensitivity to measurement errors and significantly improved data acquisition efficiency.

An MRF method may be optimized for ultrafast T₂ mapping in mouse brain using the methods of the present disclosure. The utility of this method for estimating CMRO₂ in ¹⁷O₂-inhalation study and for assessing BBB integrity in H₂¹⁷O-injection study may be validated by the direct ¹⁷O-MRI method in mice undergoing mid-cerebral artery occlusion (MCAO) surgery. BBB integrity may also be assessed using the MRF method with the co-injection of H₂¹⁷O, a T₂-shortening agent, and a gadolinium (Gd)-based contrast agent, which primarily induces T₁ shortening. The capability of simultaneous T₁ and T₂ mapping by MRF allows the determination of BBB permeability to both small (H₂¹⁷O) and large (Gd) molecules, providing more comprehensive evaluation of BBB structure.

In one configuration, indirect detection of H₂¹⁷O at the spatial resolution offered by ¹H-MRI is provided. The 1H-based method may enable the translation of the present imaging methods to low-field clinical scanners for improved patient evaluation and selection for thrombolytic therapy. Applying the ¹⁷O-MRI methods to metabolic assessment in acute stroke, in combination with diffusion and perfusion MRI, can lead to better penumbra delineation than perfusion-diffusion mismatch alone, and monitoring water exchange across BBB may allow more sensitive detection of subtle changes in BBB function. Tissue survival and vascular remodeling in post-stroke brain may be assessed with the methods of the present disclosure.

In one configuration, the methods of the present disclosure may be used to address unmet needs in stroke imaging, such as more accurate delineation of the penumbra that can better predict long-term outcome and treatment effects, sensitive detection of early-stage BBB impairment that can better predict the risk of hemorrhagic transformation, and the like. Despite its efficacy, only 3 to 5% of stroke patients are eligible to receive rtPA treatment because of the extremely short time window approved by FDA. The imaging methods of the present disclosure may provide a comprehensive assessment of the risk and benefit of thrombolytic therapy, thus leading to refined patient selection for the treatment. Such an approach may benefit a larger population of stroke patients by extending the use of rtPA beyond the current 3-hour time window. It can also provide additional guidance for future treatment in the subacute and chronic stages.

The present disclosure has described one or more preferred embodiments, and it should be appreciated that many equivalents, alternatives, variations, and modifications, aside from those expressly stated, are possible and within the scope of the invention.

1. A method for ¹⁷O imaging using a magnetic resonance imaging (MRI) system comprising:
a) administering ¹⁷O to a subject;
b) acquiring MR data for a region of interest containing the ¹⁷O within the subject using the MRI system;
c) applying a k-space filter to the MR data to create filtered MR data by:
i) segmenting k-space into at least one region;
ii) determining a number of spokes for the at least one k-space region based upon a radial sampling pattern;
iii) determining a spoke spacing for the number of spokes in the radial sampling pattern using a golden-ratio; and
d) reconstructing an MR image of the ¹⁷O within the subject using the filtered MR data.

2. The method of claim 1 wherein determining a number of spokes includes using a Fibonacci sequence to uniformly distribute the spokes in the at least one k-space region.

3. The method of claim 2 wherein the number of spokes is determined for at least two k-space regions and the Fibonacci sequence determines a different number of spokes for a second k-space region than the number of spokes in a first k-space region.

4. The method of claim 1 wherein the number of spokes in the at least one k-space region fulfills a Nyquist sampling criterion.

5. The method of claim 1 wherein the k-space filter is applied to a 2D region of k-space and wherein segmenting k-space into at least one region includes defining the 2D region of k-space as a ring.

6. The method of claim 5 wherein the spoke spacing is determined by an azimuth angle spacing divided by the golden-ratio.

7. The method of claim 5 wherein segmenting k-space includes segmenting k-space into 5 ring regions.

8. The method of claim 7 wherein the number of spokes for the 5 ring regions include 21, 34, 55, 89, or 144.

9. The method of claim 1 wherein the k-space filter is applied to a 3D region of k-space and wherein segmenting k-space into at least one region includes defining the 3D region of k-space as a sphere.

10. The method of claim 9 wherein the spoke spacing is determined by using both an azimuth angle spacing divided by the golden-ratio and a polar angle spacing divided by the golden-ratio.

11. The method of claim 1 wherein reconstructing an MR image includes regridding and Fourier transforming the filtered MR data.

12. The method of claim 1 further comprising a temporal resolution achieved by performing the method within a temporal response window of ¹⁷O.

13. The method of claim 1 wherein the ¹⁷O is administered to the subject via enriched water or oxygen gas.

14. The method of claim 1 wherein reconstructing the MR image includes reconstructing a dynamic MR image series.

15. The method of claim 1 wherein acquiring the MR data includes sampling k-space using a radially sampling trajectory.

16. A method for contrast agent imaging using a magnetic resonance imaging (MRI) system comprising:
a) administering a contrast agent to a subject;
b) acquiring MR data for a region of interest containing the contrast agent within the subject using the MRI system;
c) applying a k-space filter to the MR data to create filtered MR data by:
i) segmenting k-space into at least one region;
ii) determining a number of spokes for the at least one k-space region based upon a radial sampling pattern;
iii) determining a spoke spacing for the number of spokes in the radial sampling pattern using a golden-ratio; and
d) reconstructing an MR image of the contrast agent within the subject using the filtered MR data.

17. The method of claim 16 wherein determining a number of spokes includes using a Fibonacci sequence to uniformly distribute the spoke in the at least one k-space region.

18. The method of claim 17 wherein the number of spokes is determined for at least two k-space regions and the Fibonacci sequence determines a different number of spokes for a second k-space region than the number of spokes in a first k-space region.

19. The method of claim 16 wherein the number of spokes in the at least one k-space region fulfills a Nyquist sampling criterion.

20. The method of claim 16 wherein the k-space filter is applied to a 2D region of k-space and wherein segmenting k-space into at least one region includes defining the 2D region as a ring.

21. The method of claim 20 wherein the spoke spacing is determined by an azimuth angle spacing divided by the golden-ratio.

22. The method of claim 20 wherein segmenting k-space includes segmenting k-space into 5 ring regions.

23. The method of claim 22 wherein the number of spokes for the 5 ring regions include 21, 34, 55, 89, or 144.

24. The method of claim 16 wherein the k-space filter is applied to a 3D region of k-space and wherein segmenting k-space into at least one region includes defining the 3D region as a sphere.

25. The method of claim 24 wherein the spoke spacing is determined by using both an azimuth angle spacing divided by the golden-ratio and a polar angle spacing divided by the golden-ratio.

26. The method of claim 16 wherein reconstructing an MR image includes regridding and Fourier transforming the filtered MR data.

27. The method of claim 16 further comprising a temporal resolution achieved by performing the method within a temporal response window of the contrast agent.

28. The method of claim 16 wherein the contrast agent includes ¹⁷O.

29. A system for contrast agent imaging comprising:
a) a contrast agent administered to a subject;
b) a magnetic resonance imaging (MRI) system configured to acquire MR data for a region of interest containing the contrast agent within the subject;
c) a computer system configured to apply a k-space filter to the MR data to create filtered MR data by:
i) segmenting k-space into at least one region;
ii) determining a number of spokes for the at least one k-space region based upon a radial sampling pattern;
iii) determining a spoke spacing for the number of spokes in the radial sampling pattern using a golden-ratio; and
d) reconstructing an MR image of the contrast agent within the subject using the computer system and the filtered MR data.
